(19) United States
(12) Patent Application Publication
(10) Pub. No.: US 2002/0081707 A1
(43) Pub. Date: Jun. 27, 2002

(54) Methods for helper-dependent adenoviral vector production

(76) Inventor: Donna Armentano, Belmont, MA (US)

Correspondence Address: Steven R. Lazar, GENZYME CORPORATION, One Kendall Square, Cambridge, MA, 02319, US

(21) Appl. No.: 09/851,446

(22) Filed: May 8, 2001

Related U.S. Application Data
(60) Provisional application No. 60/215,047, filed on Jun. 29, 2000.
(60) Provisional application No. 60/259,748, filed on Jan. 4, 2001.

(51) Int. Cl. C07H 21/04; C12N 7/01; C12N 15/63; C12N 5/02

(52) U.S. Cl. 435/235.1; 536/23.72; 435/325; 435/320.1

(57) ABSTRACT

The present invention provides methods and materials for the production of helper-dependent adenovirus, such as PAV, at high titers. In one embodiment, the invention comprises methods for producing high titers of helper-dependent adenovirus comprising co-transfecting a cell permissive for production of adenovirus with: (a) a helper-dependent adenoviral vector comprising inverted terminal repeats (ITRs) and packaging sequence derived from a first adenoviral serotype, and a transgene of interest flanked by said ITRs; and (b) a chimeric, packaging-deficient helper adenovirus which contains adenoviral genes derived from the first adenoviral serotype, packaging sequence derived from a second adenoviral serotype, and ITRs derived from either the first or second adenoviral serotypes; and collecting virions produced thereby.

### BACKGROUND OF THE INVENTION

Adenoviral vectors for use in gene transfer to cells and especially in gene therapy applications, commonly are derived from adenoviruses by deletion of the early region 1 (E1) genes (Berkner, K. L., Curr. Top. Micro. Immunol. 158:39-66, 1992). Deletion of E1 genes renders such adenoviral vectors replication defective and significantly reduces expression of the remaining viral genes present within the vector. However, it is believed that the presence of the remaining viral genes in adenoviral vectors can be deleterious to the transfected cell for one or more of the following reasons: (1) stimulation of a cellular immune response directed against expressed viral proteins, (2) cytotoxicity of expressed viral proteins, and (3) replication of the vector genome leading to cell death.

One solution to this problem has been deleted adenoviral vectors, which are adenoviral vectors derived from the genome of an adenovirus containing minimal cis-acting nucleotide sequences required for the replication and packaging of the vector genome and which can contain one or more transgenes (See, U.S. Pat. No. 5,882,877 which covers pseudoadenoviral (“PAV”) or gutless vectors and methods for producing PAV, incorporated herein by reference). Such PAV vectors, which can accommodate up to 36 kb of foreign nucleic acid, are advantageous because the carrying capacity of the vector is optimized, while the potential for host immune responses to the vector or the generation of replication-competent viruses is reduced. Optimally, PAV vectors contain the 5′ inverted terminal repeat (ITR) and the 3′ ITR nucleotide sequences that contain the origin of replication, and the cis-acting nucleotide sequence required for packaging of the PAV genome, but do not comprise coding sequence for any adenoviral genes, and can accommodate one or more transgenes with appropriate regulatory elements.

Adenoviral vectors, including PAV, have been designed to take advantage of the desirable features of adenovirus which render it a suitable vehicle for nucleic acid transfer to recipient cells. Adenovirus is a non-enveloped, nuclear DNA virus with a genome size of about 36 kb, which has been well-characterized through studies in classical genetics and molecular biology (Horwitz, M. S., “Adenoviridae and Their Replication,” in Virology, 2nd edition, Fields et al., eds., Raven Press, New York, 1990). The viral genes are classified into early (designated E1-E4) and late (designated L1-L5) transcriptional units, referring to the generation of two temporal classes of viral proteins. The demarcation between these events is viral DNA replication. The human adenoviruses are divided into numerous serotypes (approximately 47, numbered accordingly and classified into 6 subgroups: A, B, C, D, E and F), based upon properties including hemagglutination of red blood cells, oncogenicity, DNA base and protein amino acid compositions and homologies, and antigenic relationships.

Recombinant adenoviral vectors have several advantages for use as gene transfer vectors, including tropism for both dividing and non-dividing cells, minimal pathogenic potential, ability to replicate to high titer for preparation of vector stocks, and the potential to carry large inserts (Berkner, K. L., Curr. Top. Micro. Immunol. 158:39-66, 1992; Jolly, D., Cancer Gene Therapy 1:51-64, 1994).

The cloning capacity of an adenovirus vector is proportional to the size of the adenovirus genome present in the vector. For example, a cloning capacity of about 8 kb can be created from the deletion of certain regions of the virus genome dispensable for virus growth, e.g., E3, and the deletion of a genomic region such as E1 whose function may be restored in trans from 293 cells (Graham, F. L., J. Gen. Virol. 36:59-72, 1977) or A549 cells (Imler et al., Gene Therapy 3:75-84, 1996). Such E1-deleted vectors are rendered replication-defective. The upper limit of vector DNA capacity for optimal carrying capacity is about 105%-108% of the length of the wild-type genome. Further adenovirus genomic modifications are possible in vector design using cell lines which supply other viral gene products in trans, e.g., complementation of E2a (Zhou et al., J. Virol. 70:7030-7038, 1996), complementation of E4 (Krougliak et al., Hum. Gene Ther. 6:1575-1586, 1995; Wang et al., Gene Ther. 2:775-783, 1995), or complementation of protein IX (Caravokyri et al., J. Virol. 69:6627-6633, 1995; Krougliak et al., supra).

Maximal carrying capacity can be achieved using adenoviral vectors deleted for most or all viral coding sequences, including PAVs (U.S. Pat. No. 5,882,877; Kochanek et al., Proc. Natl. Acd. Sci. USA 93:5731-5736, 1996; Parks et al., Proc. Natl. Acad. Sci. USA 93:13565-13570, 1996; Lieber et al., J. Virol. 70:8944-8960, 1996; Fisheretal., Virology 217:11-22, 1996; PCT Publication No. WO96/33280, published Oct. 24, 1996; PCT Publication No. WO96/40955, published December 19, 1996; PCT Publication No. WO97/25446, published Jul. 19, 1997; PCT Publication No. WO95/29993, published Nov. 9, 1995; PCT Publication No. WO96/13597, published May 9, 1996; PCT Publication No. WO97/00326, published Jan. 3, 1997; and PCT Publication No. WO99/57296. All of these documents are hereby incorporated by reference).

As noted above, PAV vectors can accommodate up to 36 kb of foreign nucleic acid (U.S. Pat. No. 5,882,877). Transgenes that have been expressed to date by adenoviral vectors include inter alia p53 (Wills et al., Human Gene Therapy 5:1079-188, 1994); dystrophin (Vincent et al., Nature Genetics 5:130-134, 1993); erythropoietin (Descamps et al., Human Gene Therapy 5:979-985, 1994); omithine transcarbamylase (Stratford-Perricaudet et al., Human Gene Therapy 1:241-256, 1990; We et al., J. Biol. Chem. 271; 3639-3646, 1996); adenosine deaminase (Mitani et al., Human Gene Therapy 5:941-948, 1994); interleukin-2 (Haddada et al., Human Gene Therapy 4:703-711, 1993); al-antitrypsin (Jaffe et al., Nature Genetics 1:372-378, 1992); thrombopoietin (Ohwada et al., Blood 88:778-784, 1996) and cytosine deaminase (Ohwada et al., Hum. Gene Ther., 7:1567-1576, 1996).

The use of adenoviral vectors in gene transfer studies to date indicates that persistence of transgene expression in target cells and tissues is often transient. At least some of the limitation is due to the generation of a cellular immune response to the viral proteins which are expressed antigenically even from a replication-defective vector, triggering a pathological inflammatory response which may destroy or adversely affect the adenovirus-infected cells (Yang et al., J. Virol. 69:2004-2015, 1995; Yang et al., Proc. Natl. Acad. Sci. USA 91:4407-4411, 1994 Zsellenger et al., Hum Gene Ther. 6:457-467, 1995; Worgall et al., Hum. Gene Ther. 8:37-44, 1997; Kaplan et al., Hum. Gene Ther. 8:45-56, 1997). Because adenovirus does not integrate into the cell genome, host immune responses that destroy virions or infected cells have the potential to limit adenovirus-based gene transfer. An adverse immune response poses a serious obstacle for high dose administration of an adenoviral vector or for repeated administration (Crystal, R., Science 270:404-410, 1995).

In order to circumvent the host immune response, which limits the persistence of transgene expression, various strategies have been employed, that generally involve either the modulation of the immune response itself or the engineering of a vector that decreases the immune response. The administration of immunosuppressive agents, together with vector administration, has been shown to prolong transgene persistence (Fang et al., Hum. Gene Ther. 6:1039-1044, 1995; Kay et al., Nature Genetics 11:191-197, 1995; Zsellenger et al., Hum. Gene Ther. 6:457-467, 1995; Scaria et al., Gene Therapy 4:611-617, 1997; WO98/08541).

Modifications to genomic adenoviral sequences contained in the recombinant vector have been attempted in order to decrease the host immune response (Yang et al., Nature Genetics 7:362-369, 1994; Lieber et al., J. Virol. 70:8944-8960, 1996; Gorziglia et al., J. Virol. 70:4173-4178; Kochanek et al., Proc. Natl. Acad. Sci. USA 93:5731-5736, 1996; Fisher et al., Virology 217:11-22, 1996). The adenovirus E3 gp19K protein can complex with MHC Class I antigens and retain them in the endoplasmic reticulum, which prevents cell surface presentation and killing of infected cells by cytotoxic T-lymphocytes (CTLs) (Wold et al., Trends Microbiol. 437-443, 1994), suggesting that its presence in a recombinant adenoviral vector may be beneficial. Other adenovirus modifications have shown promise in delivering transgenes to target cells, with persistent transgene expression having resulted therefrom (see, e.g. WO98/46781, WO98/46780, and WO98/46779 and Scaria et al., J. Virol., 72:7302-7309, 1998). The lack of persistence in the expression of adenoviral vector-delivered transgenes may also be due to limitations imposed by the choice of promoter or transgene contained in the transcription unit (Guo et al., Gene Therapy 3:802-801, 1996; Tripathy et al., Nature Med. 2:545-550, 1996). Further optimization of minimal adenoviral vectors for persistent transgene expression in target cells and tissues also involves the design of expression control elements, such as promoters, which confer persistent expression to an operably linked transgene. Promoter elements, which function independently of particular viral genes to confer persistent expression of a transgene, allow the use of vectors containing reduced viral genomes.

In addition to containing the inverted terminal repeat sequences, PAV vectors also contain a cis-acting packaging sequence, normally located at the 5′ end of the wild-type adenoviral genome. The packaging sequence contains seven functional elements, identified as A repeats (Schmid et al., J. Virol. 71:3375-3384, 1997).

Production of PAV or other minimal adenoviral vectors requires the provision of adenovirus proteins in trans which facilitate the replication and packaging of a PAV genome (and inserted foreign nucleic acid) into viral vector particles for use in gene transfer. Most commonly, such genes are provided by infecting the producer cell with a helper adenovirus containing the necessary genes. However, such viruses are potential sources of contamination of the PAV vector stock during purification if they are able to replicate and be packaged into viral particles. It is advantageous, therefore, to increase the purity of a PAV stock by reducing or eliminating the production of helper viruses that contaminate the preparation. Several strategies to reduce the production of helper viruses in the preparation of PAV and other partially deleted adenoviral stocks are disclosed in U.S. Pat. No. 5,882,887, PCT application WO99/57296 and international application No. PCT/US99/03483, filed Feb. 17, 1999 all of which are hereby incorporated herein by reference. For example, the helper virus can contain mutations in the packaging sequence of its genome which prevent packaging, or may contain an oversized adenoviral genome which cannot be packaged.

Novel helper viruses which facilitate the production of pseudoadenoviral vectors (PAV) by providing essential viral proteins in trans, but which are packaging defective due to the inclusion of binding sequences for repressor proteins that prevent utilization of the packaging signals in the helper virus genome have been disclosed in PC/US99/03483, filed Feb. 17, 1999, incorporated herein by reference. The PCT application also provides PAV producer cell lines expressing such repressor proteins and to methods for the production of PAV using such helper viruses and producer cell lines.

Recently, PAV helper viruses have been described in which packaging of the helper is reduced through the use of the Cre/Lox system (Parks et al., Proc. Natl. Acad. Sci. USA 93:13565-13570, 1996). Lox sites are placed at positions flanking the Ad packaging sequences in the helper viral genome, which is produced in conventional 293 cells. For PAV production, a Cre-expressing 293 cell is employed. The helper genome can replicate and express viral genes so that the PAV genome can be packaged, but the packaging sequences are deleted from the helper through the action of the Cre protein.

However, methods of producing helper-dependent adenoviral vectors, such as PAV, have not been maximized; measurable amounts of helper virus can remain in vector preparations. In addition, current methods of PAV production are not readily scalable for larger scale commercial uses.

The present invention provides an alternative adenoviral vector system in which the helper adenovirus contains packaging elements of a different serotype than that of the recombinant helper-dependent adenoviral vector. Because of the serotype differences, the packaging sequences present in the helper-dependent adenoviral vector have reduced ability to package the helper adenovirus. Accordingly, the ability of the helper adenovirus to become encapsidated, through recombination events, is significantly reduced, and significantly reduced amount of encapsidated helper adenovirus is produced.

Novel methods of manufacturing the PAV and other helper dependent adenoviral Ad vector and an advanced vector system for use in producing PAV, both in scaleable amounts, are also provided.

### SUMMARY OF THE INVENTION

Accordingly, the present invention provides methods and materials for the production of helper-dependent adenovirus, such as PAV, at high titers. In certain embodiments, the invention comprises methods for producing high titers of helper-dependent adenovirus comprising co-transfecting a cell permissive for production of adenovirus with: (a) a helper-dependent adenoviral vector comprising inverted terminal repeats (ITRs) and packaging sequence derived from a first adenoviral serotype, and a transgene of interest flanked by said ITRs; and (b) a chimeric packaging-deficient helper adenovirus which contains adenoviral genes derived from the first adenoviral serotype, and ITRs and packaging sequence derived from a second adenoviral serotype; and collecting the virions produced thereby.

In certain preferred embodiments, the invention comprises methods for producing high titers of helper-dependent adenovirus comprising co-transfecting a cell permissive for production of adenovirus with: (a) a helper-dependent adenoviral vector comprising a ITRs and a packaging sequence derived from a first adenoviral serotype, preferably Ad2, and a transgene of interest flanked by said inverted terminal repeats (ITRs); and (b) a chimeric packaging-deficient helper adenovirus which contains adenoviral genes derived from the first adenoviral serotype, and a packaging sequence derived from a second adenoviral serotype; and collecting the virions produced thereby. In this embodiment, the serotype origin of the ITRs flanking the chimeric packaging-deficient helper adenovirus may be derived from either the first adenoviral serotype or the second adenoviral serotype, but is preferably of the first adenoviral serotype, which is preferably Ad2.

The helper-dependent adenoviral vector is preferably a deleted adenoviral vector, such as a pseudoadenoviral vector, and its ITRs and packaging sequence are preferably derived from adenoviral subgroup C, more preferably from adenoviral serotypes 2, 5, 6 or 1, and most preferably from adenoviral serotypes 2 or 5. The helper-dependent adenoviral vector may also be derived from other adenoviral subgroups.

The packaging sequence, and in certain cases, the ITRs, of the chimeric, packaging-deficient helper adenovirus are preferably derived from an adenoviral subgroup other than the subgroup from which are derived the ITRs and packaging sequence of the helper-dependent adenoviral vector, [for example, A, B, D, E or F when the helper-dependent adenoviral vector contains ITRs and packaging sequences derived from adenoviral subgroup C]. The chimeric, packaging-deficient helper adenoviruses preferably contain one or more adenoviral genes, which have been deleted from the helper-dependent adenoviral vector. The adenoviral genes are of the same adenoviral subgroup or serotype as the ITRs and packaging sequence of the helper-dependent adenoviral vector. In the preferred embodiment wherein the ITRs and packaging sequence of the helper-dependent adenoviral vector is derived from adenoviral subgroup C, the ITRs and packaging sequence of the chimeric, packaging-deficient helper adenovirus are preferably from adenoviral subgroup B or D, and most preferably from adenoviral serotype 7 or 17, respectively, while the adenoviral genes of the helper adenovirus are derived from the subgroup C. Where the ITRs and packaging sequence of the helper-dependent adenoviral vector is derived from other adenoviral subgroups, the adenoviral genes of the chimeric, packaging-deficient helper adenovirus is preferably of the same subgroup, and the ITRs and packaging sequence of the chimeric, packaging-deficient helper adenovirus is preferably selected from a second adenoviral subgroup which is distinct from that of the helper-dependent adenoviral vector. For example, if the helper-dependent adenoviral vector comprises ITRs and packaging sequence derived from subgroup B, the chimeric, packaging-deficient helper adenovirus preferably comprises ITRs and packaging sequence from a subgroup other than B [e.g., A, C, D, E or F]. Within the ITRs, the helper adenovirus preferably comprises one or more adenoviral genes of the same subgroup as the helper-dependent adenoviral vector [e.g., subgroup B]. These adenoviral genes will provide the critical elements that are missing from the helper-dependent adenoviral vector, and allow the adenoviral vector to replicate and be encapsulated.

Other embodiments of the present invention include methods for producing high titers of helper-dependent adenovirus comprising co-transfecting a cell permissive for production of adenovirus with: (a) a helper-dependent adenoviral vector comprising inverted terminal repeats (ITRs) and packaging sequence [ψ] derived from a first adenoviral serotype from subgroup C, a transgene of interest flanked by said ITRs; and (b) a chimeric helper adenovirus with packaging sequence [ψ] derived from adenoviral subgroup B or D, and adenoviral genes derived from the first adenoviral serotype; and then collecting the virions produced from the co-transfected cell. The helper-dependent adenoviral vector is preferably a deleted adenoviral vector, such as a pseudoadenoviral vector. The adenoviral serotype of the helper-dependent adenoviral vector is preferably adenovirus 2 or 5. The ITRs of the chimeric helper adenovirus is preferably derived from the first adenoviral serotype or the second adenoviral serotype.

In other embodiments, the invention comprises chimeric, packaging-deficient helper adenoviruses useful for the propagation of helper-dependent adenoviral vectors of a first adenoviral serotype. The chimeric helper adenovirus comprises adenoviral genes derived from a first adenoviral serotype, preferably of subgroup C, and packaging sequence [ψ] derived from a second adenoviral serotype, preferably of subgroup A, B, D or E, more preferably subgroup B or D. The ITRs of the chimeric helper adenovirus is preferably derived from the first adenoviral serotype or the second adenoviral serotype. In a preferred embodiment, the chimeric, packaging-deficient helper adenovirus comprises adenoviral genes derived from an adenoviral serotype selected from the group consisting of serotypes 2 and 5, and ITRs and packaging sequence derived from an adenoviral serotype selected from the group consisting of serotypes 7 and 17.

The present invention is further directed to methods for production of helper-dependent adenoviral vectors. Such helper-dependent adenoviral vectors include pseudoadenoviral (“PAV”) or “gutless” adenoviral vectors. Helper-dependent adenoviral vectors are being developed for a variety of gene therapy applications. The vectors retain the cis elements required for DNA replication and packaging such as the inverted terminal repeats (ITRs) and packaging signal ( ) but may be devoid of all other adenoviral coding regions, which may be replaced by an expression cassette of interest and “stuffer” sequences. The viral gene products required for virus growth and encapsidation must therefore be supplied in trans by a helper virus in order to produce PAV. In current schemes, both the helper-dependent virus and the helper virus are derived from the same adenovirus serotype and PAV is co-propagated with helper virus which leads to the production of both vectors within the cell. Several strategies have been employed to reduce the presence of helper in virus preparations.

One strategy is based on constructing PAV and helper with different genomic lengths such that PAV and helper virus particles can be separated by CsCl density gradient centrifugation. Another is based on modifying the packaging signals within PAV and/or the helper virus such that the helper becomes less efficiently encapsidated. A third strategy, which is the most efficient, is based on Cre/Lox mediated excision of the packaging signal from the helper in the producer cell resulting in 100-1000 fold reduction of encapsidation. All these strategies yield some helper virus contamination in the PAV preparation and tend to generate replication competent adenovirus.

Thus, in one embodiment, the present invention comprises methods for producing high titers of helper-dependent adenovirus comprising co-transfecting a cell permissive for production of adenovirus with: (1) a helper-dependent adenoviral vector comprising inverted terminal repeats (ITRs) and packaging sequence [ψ] derived from a first adenoviral subgroup, and a transgene of interest flanked by said ITRs; and (2) a packaging-deficient helper adenovirus which contains adenoviral genes derived from a first adenoviral subgroup, but packaging sequence [ψ] from a second adenoviral subgroup; and collecting virions produced thereby. The ITRs of the helper adenovirus may preferably be derived from either the first or second adenoviral subgroup.

The packaging sequence [ψ] of the helper adenoviral vector is preferably selected from the group consisting of subgroup B and subgroup D, more preferably selected serotype 7 and serotype 17, respectively. The adenoviral genes in the helper are preferably selected from subgroup C. The packaging-deficient helper virus may contain mutations in the packaging sequence of its genome which prevent packaging, or may contain an oversized adenoviral genome which cannot be packaged. Alternatively, packaging of the helper virus may reduced through the use of the Cre/Lox system or other recombinase. The ITRs of the helper adenovirus may preferably be derived from either the first or second adenoviral subgroup.

The ITRs and packaging sequence [ψ] of the helper-dependent adenoviral vector are preferably derived from adenovirus subgroup C, more preferably derived from the adenovirus serotype 2 or serotype 5.

In other embodiments, the present invention comprises methods for producing high titers of helper-dependent adenovirus comprising co-transfecting a cell permissive for production of adenovirus with both (1) a helper-dependent adenoviral vector comprising ITRs and packaging sequence [ψ] derived from a first adenoviral serotype, and a transgene flanked by said ITRs; and (2) a chimeric helper adenoviral vector comprising packaging sequence [ψ] derived from a second adenoviral serotype, adenoviral genes derived from the first adenoviral serotype, and inverted terminal repeats (ITRs) derived from either the first or second adenoviral serotype; and then collecting virions produced thereby.

In preferred embodiments of the invention, the helper-dependent adenoviral vector is a partially or fully deleted adenoviral vector. In the most preferred embodiment, the helper-dependent adenoviral vector is a fully deleted pseudoadenoviral vector. The helper-dependent adenoviral vector preferably comprises ITRs and packaging sequences [ψ] derived from adenoviral subgroups C, and more preferably the ITRs and packaging sequences [ψ] are derived from adenoviral serotypes 2, 5, 6 or 1.

The helper adenovirus useful in the methods of the present invention is preferably a chimeric adenovirus which contains a full complement of the adenoviral genome. Alternatively, the helper adenovirus may be a chimeric adenovirus which contains adenoviral genes to complement the adenoviral functions which have been deleted from the helper-dependent adenoviral vectors which the helper adenovirus is designed to support. In either case, one or more of the adenoviral genes is preferably derived from the same adenoviral subgroup as the helper-dependent adenoviral vector it is designed to support, while the packaging sequence of the helper adenovirus is derived from a second subgroup. The ITRs of the helper adenovirus are preferably derived from either the first adenoviral subgroup or the second adenoviral subgroup. In preferred embodiments, the adenovirus E1 genes in the helper are preferably from adenovirus subgroup C, and is more preferably of adenoviral serotype 2, 5, 6 or 1, most preferably derived from adenoviral serotype 2 or 5. Alternatively, or in addition, the helper adenovirus may be packaging-deficient. For example, the helper adenovirus can contain mutations in the packaging sequence of its genome which prevent packaging, or may contain an oversized adenoviral genome which cannot be packaged.

Other helper viruses useful in the present invention may be packaging defective due to the inclusion of binding sequences for repressor proteins that prevent utilization of the packaging signals in the helper virus genome, or in which packaging of the helper is reduced through the use of the Cre/lox or other recombinase system. Examples of other recombinase systems that can be used include Flp recombinase (Senecoff et al., 1985, Proc. Natl. Acad. Sci. USA 82:7270-7274; Buchholz et al., 1998, Nature Biotechnol. 16:657-662; Buchholz et al., 1996, NAR 24:4256-4262), and the phage [φ] C31 recombinase system is described in Kuhstoss and Rao, J. Mol. Biol. 222:897-908 (1991); U.S. Pat. No. 5,190,981; Groth et al., PNAS Early Edition, www.pnas.org/cgi/doi/10.1073/pnas.090527097; and PCT Patent Publication WO00/11155. There are currently approximately 105 proteins in subgroups of site specific recombinases. See generally, Nunes-Duby et al., 1998, Nucleic Acids Res. 26:391-406; Argos et al., 1986, EMBO J. 5:433-440. In addition to the above recombinases, a recombinase (R) encoded by the pSR1 plasmid of the yeast Zygosaccharomyces rouxii has similar function to FLP (Kilby et al., 1993, TIG 9:413-421). The “R” recombinase and its recognition sequences may also facilitate the binding and recombination referred to herein. The disclosure of all of these publications is hereby incorporated herein by reference.

In other embodiments, the present invention comprises helper-dependent adenoviral vectors. The helper-dependent adenoviral vectors of the present invention preferably comprise inverted terminal repeats (ITRs) and packaging sequence, and a transgene of interest flanked by said ITRs. In preferred embodiments of the invention, the helper-dependent adenoviral vector is a pseudoadenoviral vector, which contains no coding sequences for adenoviral genes. In preferred embodiments, the ITRs and packaging sequence are derived from an adenoviral serotype selected from the group consisting of adenovirus subgroup C, more preferably from adenovirus serotype 2, 5, 6 or 1, more preferably from adenovirus serotypes 2 or 5.

Thus, in certain embodiments, the present invention comprises a chimeric helper adenovirus which contains a packaging sequence [ψ] derived from a different adenoviral serotype subgroup than that of the adenoviral genome of the helper adenovirus, said packaging sequence being flanked by target sites of recombination such as lox sites, for use with the Cre/lox system. This is where the utility of using a chimeric packaging signal lies. Thus, in such embodiments of the invention, the packaging signal of the chimeric adenovirus may be flanked by lox sites. In the use of a helper adenovirus using the Cre/lox system, if there is a recombination event of a helper adenovirus with PAV or with 293 sequences in the packaging signal, it results in the loss of one of the lox sites surrounding the packaging signal. This in turn results in the failure of the packaging signal to be excised from the helper and thus during the expansion process, PAV preparations will be contaminated with helper. With the chimeric packaging signal, such recombination events, and thus, such contamination, will be greatly reduced.

Description of the Sequences:

Sequence ID NO: I is a nucleotide sequence from the ITR and y sequences of Ad serotype 2.

Sequence ID NO:2 is a nucleotide sequence from the ITR and ψ sequences of Ad serotype 4.

Sequence ID NO:3 is a nucleotide sequence from the ITR and ψ sequences of Ad serotype 7.

Sequence ID NO:4 is a nucleotide sequence from the ITR and ψ sequences of Ad serotype 12.

Sequence ID NO:5 is a nucleotide sequence from the ITR and ψ sequences of Ad serotype 17.

### BRIEF DESCRIPTION OF THE FIGURES

FIG. 1 illustrates the alignment of ITR and ψ sequences from Ad2 (subgroup C) with Ad12 (subgroup A).

FIG. 2 illustrates the alignment of ITR and ψ sequences from Ad7 (subgroup B) with Ad2 (subgroup C).

FIG. 3 illustrates the alignment of ITR and ψ sequences from Ad17 (subgroup D) with Ad2 (subgroup C).

FIG. 4 illustrates the alignment of ITR and ψ sequences from Ad4 (subgroup E) with Ad2 (subgroup C).

FIG. 5 illustrates constructs generated containing either the Ad7 or Ad17 ITRs +/−ψ sequences linked to ad Ad2 genome in which the El region was deleted and replaced with a β-galactosidase expression cassette. Ad2-p7 and Ad2-7 contain the ITRs +/−ψ sequences from Ad7, respectively and Ad2-p17 and Ad2-17 contain the ITRs +/−ψ sequences from Ad17, respectively. Ad2-EGFP is a positive control virus that is entirely derived from Ad2 in which the E1 region was deleted and replaced with a green fluorescent protein expression cassette.

FIG. 6. Panel A illustrates various assays that were conducted for analysis of viral replication and packaging. Plasmids were digested with SnaBI and the DNAs were transfected into parallel cultures of 293 cells. The ability of the constructs to replicate over a time course of 0 to 96 hours post-transfection was monitored by Southern analysis, illustrated in FIG. 6, panel B.

FIG. 7 shows the results of plaque assays. For both the Ad2-p7 and Ad2-p17 constructs virus titer is reduced more that one order of magnitude compared to positive control, pAd2EGFP. In addition, the appearance of plaques is delayed by 3-4 days.

FIG. 8 illustrates the yield of Ad2-p7 is increased by more that three orders of magnitude when cultures are co-infected with wild type Ad7 virus while the yield remains unchanged in cultures co-infected with wild type Ad2. This suggests that the wild type Ad7 virus can supply a factor(s) in trans that rescues the Ad2-p7 virus. Similar results were observed with Ad2-p 17.

FIG. 9 illustrates the relative titers of vector [Ad2-β-ga14] and chimeric helper adenovirus [Ad2-ψ17] with packaging sequence derived from Ad17.

FIG. 10 shows the alignment of Ad2 and Ad17 packaging sequences. Sequences shown in bold are the A repeats in the packaging signal. Sequences underlined are enhancer regions. One of the problems encountered in the scale up of PAV (helper dependent adenoviral vector) is recombination between the helper and PAV. This is particularly important in strageges that use a recombinase/target sequence such as the Cre-lox system to excise the packaging signal from the helper in order to reduce helper contamination. A recombination event in the ψ region would lead to loss of the ability to remove ψ from the helper. The ψ regions share approximately 74% homology and since Ad17 ψ functions in the context of an Ad2 based vector, this might be incorporated into helper vectors as one strategy to reduce recombination between helper and PAV.

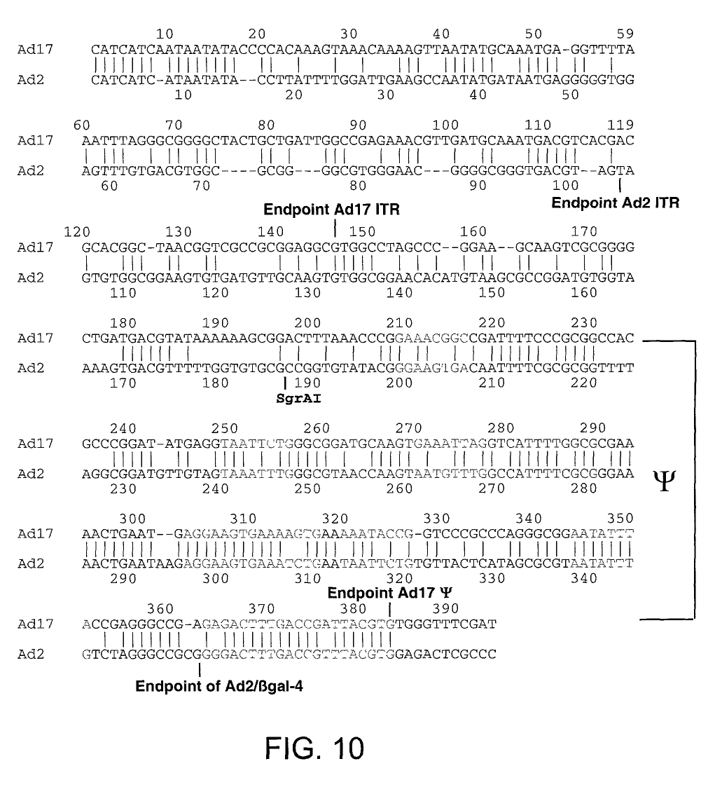

### DETAILED DESCRIPTION OF THE INVENTION

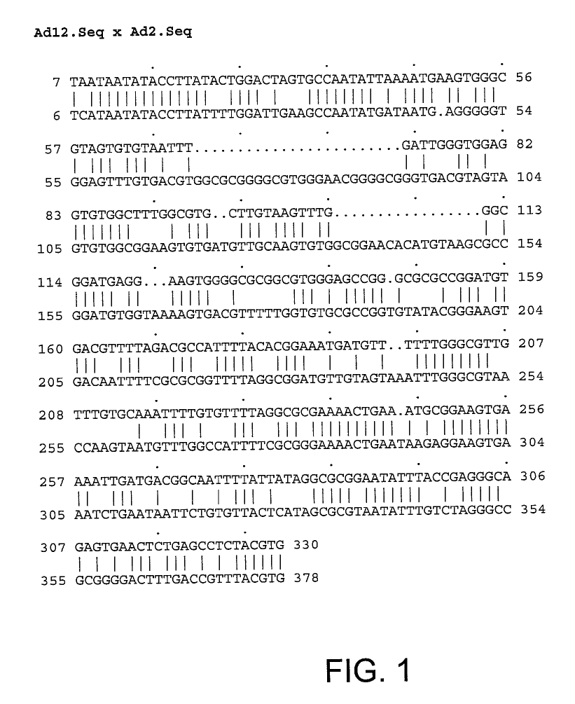

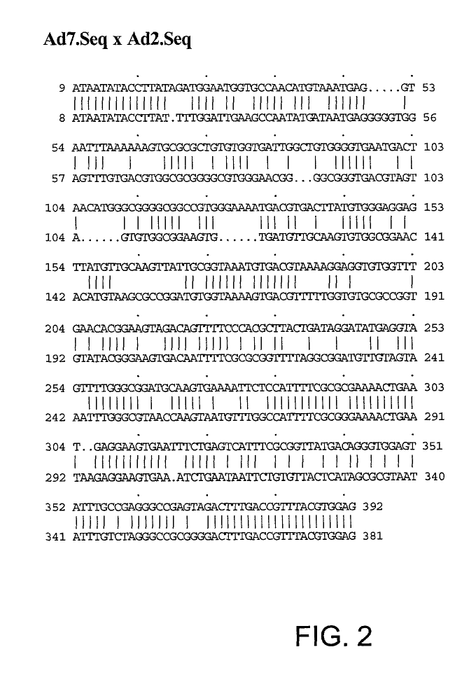

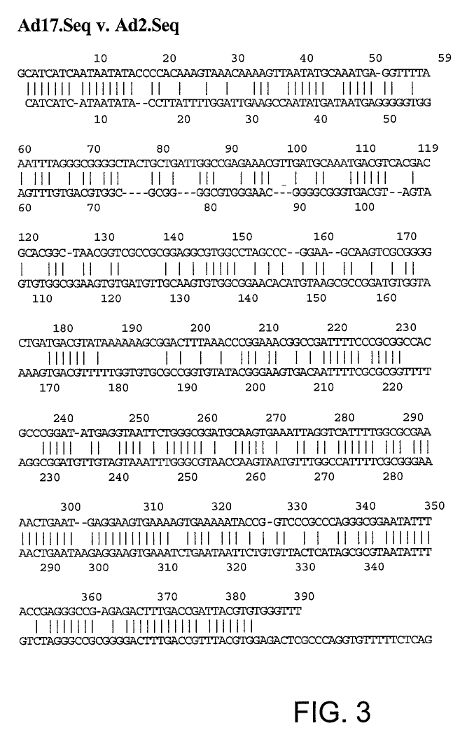

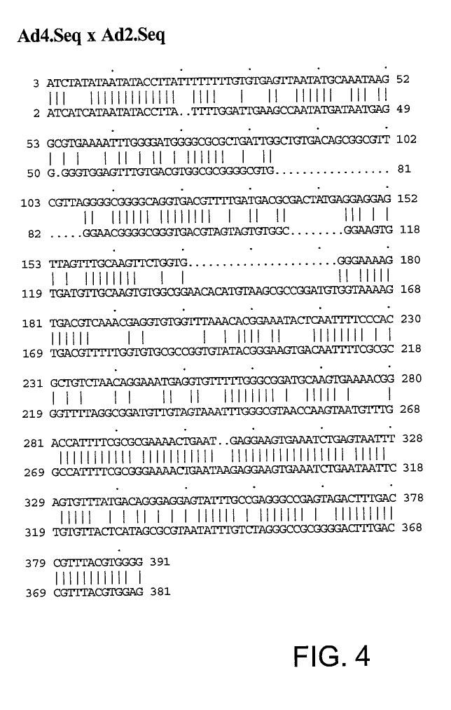

Adenovirus DNA replication is well understood and both viral and cellular components that are required for this process have been identified for different adenovirus subgroups. Adenoviral DNA encapsidation is less understood. Encapsidation or packaging signal sequences (ψ) have been identified for subgroup C viruses as well as cellular factors that bind to these sequences. Not all of the identified subgroup C packaging signal elements are conserved in viruses from other subgroups and the overall homology of the ITR and packaging signal region of subgroup C viruses with other subgroup members ranges from 60-68%. FIGS. 1-4 show the alignment of ITR and ψ sequences from Ad2 (subgroup C) with Ad12 (subgroup A), Ad7 (subgroup B), Ad17 (subgroup D) and Ad4 (subgroup E), respectively.

The invention is based on the observation that viruses from different subgroups do not efficiently cross-package each other due to differences in the required packaging signal sequences (both known and unknown) and differences in viral proteins that direct subgroup specific packaging. The invention is directed to novel helper adenoviruses for the production of helper-dependent adenoviral vectors, such as PAV. A helper vector could contain the packaging signal +/− the ITRs from one subgroup but contain the remainder of the genome of the subgroup from which PAV is derived. This would require a complementing cell line that supplies the packaging factor(s) in trans for packaging the helper. The helper can then be used in a non-complementing cell line to generate PAV. In this scenario, the helper will replicate and package PAV but packaging of the helper will be compromised.

Cell lines useful in the methods of the present invention include those cell lines which are permissive for adenoviral replication and packaging, including, but not limited to human 293 embryonic kidney cells, A549 embryonic kidney cells, and PerC6 embryonic retinal cells.

Most cell lines presently in use are derived from human 293 embryonic kidney cells, which contain an E1 adenoviral gene, ITRs and packaging sequence derived from the adenovirus 2 serotype. In order to reduce the potential for recombination between a helper adenovirus and the E1 cell line to generate unwanted replication-competent adenovirus, it is preferred that the chimeric helper adenovirus of the present invention comprise packaging sequences from a serotype other than adenovirus 2 serotype. In addition, the ITRs of the chimeric helper adenovirus may preferably be derived from a serotype other than adenovirus 2 serotype.

The invention is also directed at generating helper vectors that have a reduced potential for recombination with PAV. ITRs on the helper and PAV can be derived from different subgroups to reduce the potential for recombination. As shown in FIGS. 1-4 the homologies of ITRs between subgroups ranges from 60-80%.

The invention described and claimed herein is not to be limited in scope by the specific embodiments herein disclosed, since these embodiments are intended only as illustrations of several aspects of the invention. Any equivalent embodiments are intended to be within the scope of this invention. Indeed, various embodiments and modifications of the invention, in addition to those shown and described herein will become apparent to those skilled in the art from the foregoing description and the examples which follow. Such modifications also fall within the scope of the appended claims. Various references and publications are cited within this specification, and the disclosures of all of which are hereby incorporated herein by reference in their entireties.

### EXAMPLES

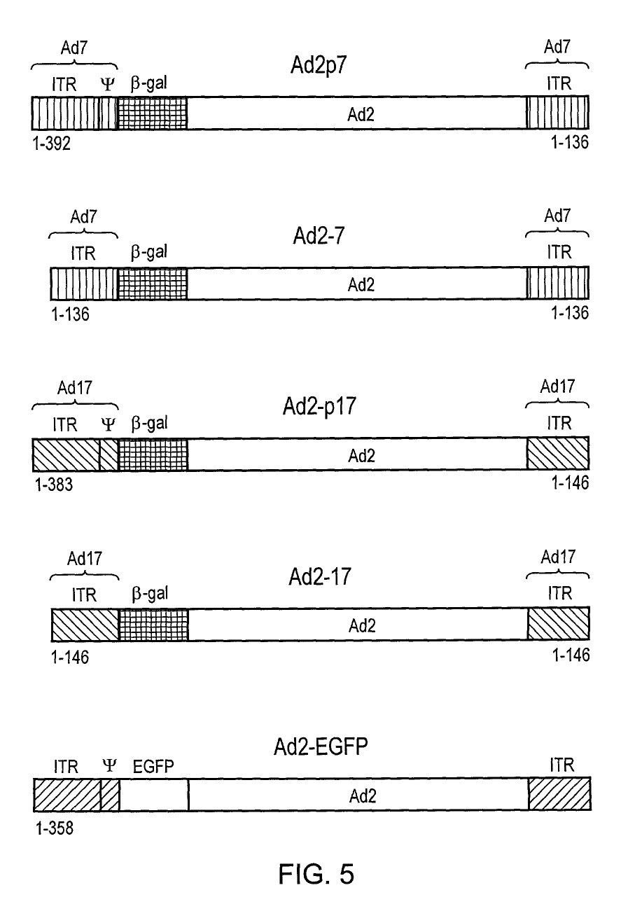

Chimeric first generation adenovirus vectors were constructed to determine if the ITRs and ψ from other subgroups would allow replication and packaging of an otherwise Ad2 genome. FIG. 5 depicts constructs that were generated containing either the Ad7 or Ad17 ITRs +/−ψ sequences linked to an Ad2 genome in which the E1 region was deleted and replaced with a β-galactosidase expression cassette. Ad2-p7 and Ad2-7 contain the ITRs +/−ψ sequences from Ad7, respectively and Ad2-p17 and Ad2-17 contain the ITRs +/−ψ sequences from Ad17, respectively. Ad2-EGFP is a positive control virus that is entirely derived from Ad2 in which the E1 region was deleted and replaced with a green fluorescent protein expression cassette. All vectors contain a 2.9kb deletion in the E3 region. The constructs were generated in plasmid form from which the chimeric genomes could be excised by digestion with restriction endonuclease SnaBI.

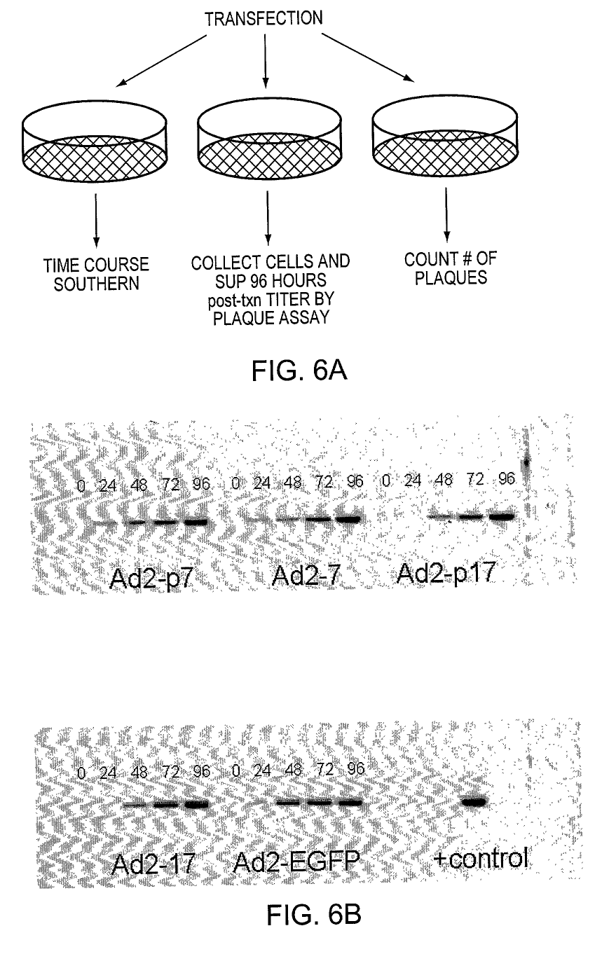

FIG. 6, panel A schematically depicts the various assays that were done for analysis of viral replication and packaging. Plasmids were digested with SnaBI and the DNAs were transfected into parallel cultures of 293 cells. The ability of the constructs to replicate over a time course of 0 to 96 hours post-transfection was monitored by Southern analysis, shown in FIG. 6, panel B. DNA replication of Ad2-p7 and Ad2-7 appear to exhibit similar kinetics to the positive control vector, Ad2-EGFP, indicating that the Ad2 (subgroup C) replication machinery can replicate DNA containing ITRs derived from Ad7 (subgroup B). DNA replication of Ad2-p17 and Ad2-17 appears to be delayed and is not detected until 48 hours post transfection. By later time points however, the DNAs accumulate to similar amounts as Ad2-EGFP indicating that although delayed, the Ad2 replication machinery can replicate DNA containing ITRs derived from Ad17 (subgroup D. Constructs that do not contain a packaging signal appear to replicate with similar kinetics to their counterparts that do suggesting that the packaging signal is dispensable for DNA replication.

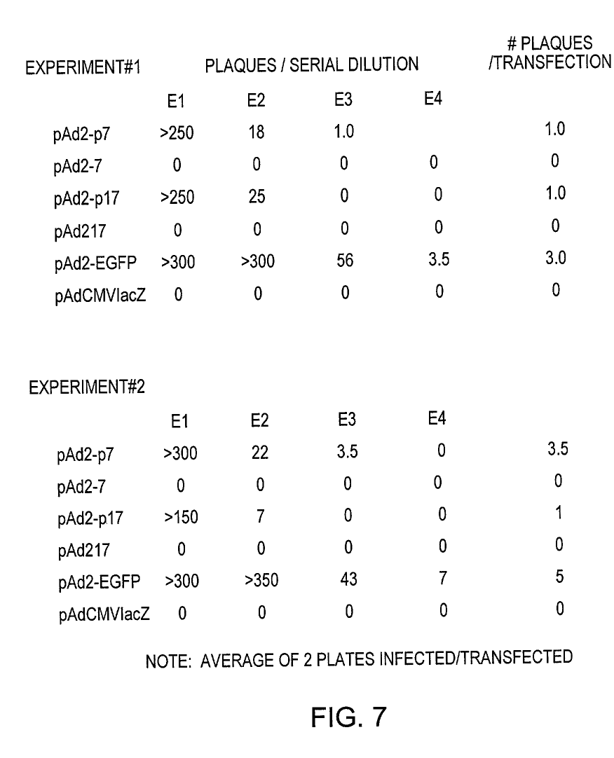

Cultures harvested at 96 hours post-transfection were subjected to three freeze-thaw cycles and the released virus was titered by plaque assay. The results of each experiment shown in FIG. 7 represent the averages from duplicate samples. For both the Ad2-p7 and Ad2-p17 constructs virus titer is reduced more that one order of magnitude. In addition, the appearance of plaques is delayed by 3-4 days. This indicates that while the DNAs can be replicated, they are not efficiently being incorporated into virus particles. This suggests that a subgroup specific factor(s) might be involved packaging signal recognition and that the Ad2 factors less efficiently package genomes containing packaging signals derived from other subgroups. This phenomenon would be reflected in reduced titers and delayed plaque formation.

Cultures were also overlayed following transfection and the number of plaques was scored.

This was done as a control for transfection efficiency. While the number of plaques obtained was lower for Ad2-p7 and Ad2-p17 the differences in virus yield cannot be accounted for by this variation. In addition, the appearance of plaques was delayed for these constructs compared to the Ad2-EGFP control virus.

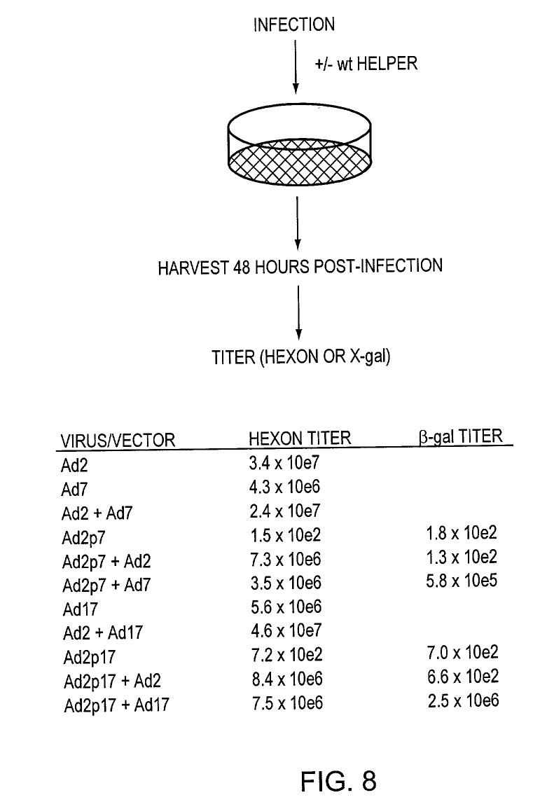

In order to determine if a subgroup specific factor(s) was involved in packaging, rescue experiments were performed. Titered Ad2-p7 and Ad2-p17 virus was used to infect 293 cells either alone or with wild type virus (either Ad2 or the serotype from which the ITRs were derived). Forty-eight hours post-infection the cultures were harvested and were subjected to three freeze-thaw cycles. The released virus was titered by hexon staining to measure total virus yield and by X-gal staining to measure chimeric virus yield. As shown in FIG. 8, the yield of Ad2-p7 is increased by more that three orders of magnitude when cultures are co-infected with wild type Ad7 virus while the yield remains unchanged in cultures co-infected with wild type Ad2. This suggests that the wild type Ad7 virus can supply a factor(s) in trans that rescues the Ad2-p7 virus. A similar result is observed for Ad2-p17. The yield of this virus is also increased by more than three orders of magnitude when cultures are co-infected with wild type Ad17 whereas the yield remains unchanged in cultures co-infected with wild type Ad2. This suggests that wild type Ad17 virus can supply a factor(s) in trans that rescues the Ad2-p 17 virus.

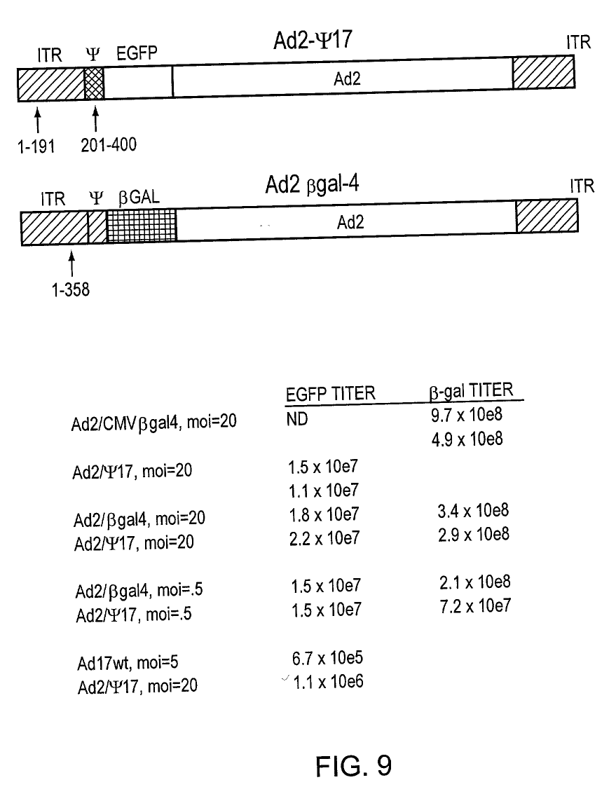

In order to determine if the packaging signal (ψ) directs subgroup specific packaging, a construct was generated that is solely derived from Ad2 except for ψ. Ad2-ψ17, shown in panel A, is Ad2-based but contains ψ from Ad 17 and was generated by transfection into 293 cells. This virus was expanded in PerC.6 cells and analyzed for virus yield in the presence or absence of wild type Ad17. As shown, the titer of Ad2-ψ17, unlike Ad2-p17 (FIG. 9), does not increase when it is grown in the presence of wild type Ad17. This suggests that supplying Ad17 functions in trans does not increase titer and that elements involved in subgroup specific packaging lie outside of the ψ region. The yield of Ad2-ψ17 compared to Ad2/βgal-4 which is completely Ad2-based is modestly affected suggesting that the Ad17 ψ can function in place of Ad2 ψ, but less efficiently. The titer of Ad2-ψ17 is not affected when grown in the presence of Ad2/βgal-4, further supporting the interchangeability of the ψ regions.

From the above, it can be concluded that packaging of adenovirus is subgroup specific, and that elements involved in subgroup specific packaging lie outside of the conventional the ψ regions. Thus, incorporation of non-Ad2 ψ sequences into helper vectors for use with helper dependent vectors derived from Ad2, such as PAV, may be a useful strategy for reducing recombination between helper and PAV in the scale-up process. This is particularly important in strategies that use a recombinase/target sequence such as the Cre-lox system to excise the packaging signal from the helper in order to reduce helper contamination. A recombination event in the ψ region would lead to loss of the ability to remove ψ from the helper.

1. A method for producing high titers of helper-dependent adenovirus comprising:
a. co-transfecting a cell permissive for production of adenovirus with:
1. a helper-dependent adenoviral vector comprising inverted terminal repeats (ITRs) and packaging sequence derived from a first adenoviral serotype, and a transgene of interest flanked by said ITRs; and
2. a chimeric, packaging-deficient helper adenovirus which contains adenoviral genes derived from the first adenoviral serotype, packaging sequence derived from a second adenoviral serotype, and ITRs derived from either the first or second adenoviral serotype; and
b. collecting virions produced thereby.

2. The method of claim 1, wherein the helper-dependent adenoviral vector is a pseudoadenoviral vector.

3. The method of claim 1, wherein the packaging sequence of the helper adenovirus derived from adenoviral subgroup B or D.

4. The method of claim 1, wherein the first adenoviral serotype is from subgroup C.

5. The method of claim 4, wherein the first adenoviral serotype is adenovirus 2.

6. The method of claim 4, wherein the second adenoviral serotype is selected from the group consisting of subgroup B and subgroup D.

7. The method of claim 5, wherein the second adenoviral serotype is selected from the group consisting of adenovirus 7 and adenovirus 17.

8. A method for producing high titers of helper-dependent adenovirus comprising:
a. co-transfecting a cell permissive for production of adenovirus with:
1. a helper-dependent adenoviral vector comprising inverted terminal repeats (ITRs) and packaging sequence derived from a first adenoviral serotype from subgroup C, a transgene of interest flanked by said ITRs; and
2. a chimeric, helper adenovirus with adenoviral genes derived from the first adenoviral serotype, packaging sequence derived from adenoviral subgroup B or D, and ITRs derived from either the first adenoviral serotype or subgroup B or D; and
b. collecting virions produced thereby.

9. The method of claim 8, wherein the helper-dependent adenoviral vector is a pseudoadenoviral vector.

10. The method of claim 8, wherein the first adenoviral serotype is selected from the group consisting of adenovirus 2 and adenovirus 5.

11. A chimeric, packaging-deficient helper adenovirus useful for the propagation of helper-dependent adenoviral vectors of a first adenoviral subgroup, wherein the helper adenovirus comprises adenoviral genes derived from the first adenoviral subgroup, packaging sequence [ψ] derived from a second adenoviral subgroup, and ITRs derived from either the first or second adenoviral subgroup.

12. The chimeric, packaging-deficient helper adenovirus of claim 11, wherein the packaging sequence is derived from an adenoviral subgroup selected from subgroup A, B, D or E, and the adenoviral genes are derived from an adenovirus of adenoviral subgroup C.

13. The chimeric, packaging-deficient helper adenovirus of claim 11, wherein the packaging sequence is derived from adenoviral serotype 5, and the adenoviral genes are derived from an adenoviral serotype selected from the group consisting of serotypes 7 and 17.

14. The chimeric, packaging-deficient helper adenovirus of claim 11, wherein the packaging sequence is derived from adenoviral serotype 2, and the adenoviral genes are derived from an adenoviral serotype selected from the group consisting of serotypes 7 and 17.

15. The chimeric, packaging-deficient helper adenovirus of claim 11, wherein the packaging sequence is flanked by recombinase recognition sites.
